(19) United States
(12) Patent Application Publication
(10) Pub. No.: US 2025/0032189 A1
(43) Pub. Date: Jan. 30, 2025

(54) METHODS AND SYSTEMS FOR GENERATING 3D MODELS OF EXISTING BONE TUNNELS FOR SURGICAL PLANNING

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Alyssa ANDRADE, Dedham, MA (US); Brian William QUIST, Salem, NH (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/768,484

(22) Filed: Jul. 10, 2024

Related U.S. Application Data
(60) Provisional application No. 63/515,628, filed on Jul. 26, 2023.

(51) Int. Cl. A61B 34/10; A61B 17/32

(52) U.S. Cl. CPC A61B 34/10; A61B 17/32; A61B 2017/320056; A61B 2034/102; A61B 2034/105

(57) ABSTRACT

Some examples are directed to methods and related systems for generating 3D models of existing bone tunnels for surgical planning.

### CROSS-REFERENCE TO RELATED APPLICATION

This application claims the benefit of priority to U.S. Provisional Patent Application No. 63/515,628, titled “Methods and Systems For Generating 3D Models of Existing Bone Tunnels For Surgical Planning,” filed Jul. 26, 2023, which is incorporated herein by reference in its entirety.

### BACKGROUND

The anterior cruciate ligament (ACL) serves as the primary mechanical restraint in the knee to resist anterior translation of the tibia relative to the femur. Similarly, the posterior cruciate ligament (PCL) serves as a mechanical restraint to resist posterior translation of the tibia relative to the femur. These cruciate ligaments contribute significantly to knee stability, and ACL injury is quite common. Most ACL injuries are complete tears of the ligament.

As ACL injuries occur often in patients that are young and active, reconstruction of the ACL is performed to enable a return to activity. The goal is to restore stability of the knee and reduce the chances of further damage to the meniscus and articular cartilage that may lead to degenerative osteoarthritis. Reconstruction may consist of placement of a substitute graft (e.g., autograft from either the central third of the patellar tendon or the hamstring tendons). The ends of the graft are placed into respective tunnels prepared through the femur and the tibia. The ends of the graft may be attached using hardware such as interference screws or a suspensory fixation device like the ENDOBUTTON™ brand fixation devices manufactured by Smith & Nephew of Andover, Massachusetts, USA.

One challenge in ACL reconstruction is where the tunnels should be placed. The native ACL consists of 2 major bundles—the anteromedial (AM) and the posterolateral (PL) bundle. Often, the goal of the surgery is to place the reconstruction in an anatomical location, for example, placing a single tunnel within the footprint of the native ACL attachment site. In other cases, reconstruction may involve creating two tunnels in both the femur and tibia in an attempt to recreate the two native bundles.

The failure rate in ACL reconstructions ranges from 10-15%, with 61% of the failures attributable to technical errors, such as errors in actual-tunnel location relative to planned-tunnel location. For example, some 80% of technical failures are femoral tunnel malposition and 37% are tibial tunnel malposition. Where a first—or “primary”-ACL reconstruction fails, a procedure known as a revision ACL reconstruction may be attempted. However, unlike with a primary ACL reconstruction, planning and execution of a revision ACL reconstruction must take into account the existence of tunnels and hardware that had been put in place during the primary ACL reconstruction.

A user such as a surgeon may use cross-sectional images such as MRI (Magnetic Resonance Imaging) images and/or CT (Computed Tomography) images to locate an existing tunnel or tunnels in the bone, and to characterize the tunnel(s) at least in terms of length, cross-sectional shape/diameter, and orientation. However, while an existing tunnel, when initially formed, may have been cylindrical or otherwise generally uniform in cross-sectional shape and size, over time such a tunnel may undergo non-uniform bone in-growth and thus may become increasingly difficult to effectively characterize.

Known techniques for measuring tunnel widening after ACL reconstruction may be considered adaptable for locating and characterizing existing tunnels for revision ACL construction. However, such known techniques may require that combinations of different software packages be used at different stages. This, in turn, may require that users expend time and expertise manually importing and exporting content to and from the software packages. Also, some of the known techniques may require that a user subjectively choose sample image data from an overall body of available image data for processing, and/or may involve using software packages that regard important image data points as mere outliers. Furthermore, some of the known techniques may require that users subjectively manipulate contents of multiple cross-sectional images before such images can be considered suited for downstream processing. Improvements for efficiently and accurately characterizing existing bone tunnels are therefore desirable.

### SUMMARY

One example is a processor-implemented method comprising: receiving, by processing structure, a 3D point cloud representing a bone tunnel, wherein each point of the point cloud has an (x,y,z) coordinate in a coordinate system; processing, by processing structure, the points of the point cloud to determine at least a first principal axis of the point cloud in the coordinate system; determining, by processing structure, endpoints of the point cloud based on the first principal axis; projecting, by processing structure, each of the points of the point cloud onto a 2D plane that is perpendicular to the first principal axis thereby to generate projected points; determining, by processing structure, an outermost periphery of a first enclosed N-sided polygon in the 2D plane centered on the first principal axis and enclosing the projected points; generating, by processing structure, a 3D object model based at least on the outermost periphery, the first principal axis, and the endpoints; and providing, by processing structure, the 3D object model to a surgical planning application.

The example method may comprise, prior to the projecting, rotating, by processing structure, each point of the point cloud to align the first principal axis of the point cloud with a z axis of the coordinate system, wherein the 2D plane that is perpendicular to the first principal axis is an x-y plane in the coordinate system.

The example method may comprise determining, by processing structure, an innermost periphery corresponding to a second enclosed N-sided polygon in the 2D plane that is centered on the first principal axis and enclosed by the projected points, wherein generating the 3D object model is additionally based on the innermost periphery.

The example method may comprise displaying, by the surgical planning application, the 3D object model in association with a 3D tunnel model of the bone tunnel.

The example method may comprise, prior to receiving the point cloud: segmenting, by processing structure, tunnel features in each of a plurality of cross-sectional images of a bone; generating, by processing structure, a 3D tunnel model based on the segmenting; and generating, by processing structure, the point cloud based on the 3D tunnel model.

The segmenting the tunnel features may comprise generating, by processing structure, a mask representing the tunnel features for each of a plurality of the cross-sectional images.

The example method may comprise receiving, by processing structure, a 3D tunnel model representing the bone tunnel; and generating, by processing structure, the point cloud based on the 3D tunnel model.

In the example method, the first enclosed N-sided polygon may be a first circle. In the example method, the first circle may be a best-fit circle.

In the example method, the first circle may be larger in diameter by a tolerance amount than a best-fit circle.

In the example method, generating the 3D cylinder model may be additionally based on the best-fit circle.

In the example method, the surgical planning application may be configurable to characterize the 3D object model as at least a portion of a no-drilling zone.

In the example method, the surgical planning application may be configurable to characterize the 3D object model as at least a portion of a drilling zone.

In the example method, the 3D cylinder model may contain a plurality of peripheries including the outermost periphery.

Yet another example is device comprising: processing structure; and a memory coupled to the processing structure, the memory storing instructions that, when executed by the processing structure, cause the processing structure to: receive a 3D point cloud representing a bone tunnel, wherein each point of the point cloud has an (x,y,z) coordinate in a coordinate system; process the points of the point cloud to determine at least a first principal axis of the point cloud in the coordinate system; determine endpoints of the point cloud based on the first principal axis; project each of the points of the point cloud onto a 2D plane that is perpendicular to the first principal axis thereby to generate projected points; determine an outermost periphery of a first enclosed N-sided polygon in the 2D plane centered on the first principal axis and enclosing the projected points; generate a 3D object model based at least on the outermost periphery, the first principal axis, and the endpoints; and provide the 3D object model to a surgical planning application.

In the example device, the memory may store instructions that, when executed by the processing structure, cause the processing structure to: rotate each point of the point cloud to align the first principal axis of the point cloud with a z axis of the coordinate system, wherein the 2D plane that is perpendicular to the first principal axis is an x-y plane in the coordinate system.

In the example device, the memory may store instructions that, when executed by the processing structure, cause the processing structure to: determine an innermost periphery corresponding to a second enclosed N-sided polygon in the 2D plane that is centered on the first principal axis and enclosed by the projected points, wherein generating the 3D object model is additionally based on the innermost periphery.

In the example device, the processing structure may be coupled to a display device and the memory may store instructions when executed by the processing structure, cause the processing structure to: display, on the display device, the 3D object model in association with a 3D tunnel model of the bone tunnel.

In the example device, the memory may store instructions that, when executed by the processing structure, cause the processing structure to: segment tunnel features in each of a plurality of cross-sectional images of a bone; generate a 3D tunnel model based on the segmenting; and generate the point cloud based on the 3D tunnel model.

In the example device, the memory may store instructions that, when executed by the processing structure, cause the processing structure to: generate a mask representing the tunnel features for each of a plurality of the cross-sectional images.

In the example device, the memory may store instructions that, when executed by the processing structure, cause the processing structure to: receive a 3D tunnel model representing the bone tunnel; and generate the point cloud based on the 3D tunnel model.

In the example device, the first enclosed N-sided polygon may be a first circle.

In the example device, the first circle may be a best-fit circle.

In the example device, the first circle may be larger in diameter by a tolerance amount than a best-fit circle.

In the example device, generating the 3D cylinder model is additionally based on the best-fit circle.

In the example device, the memory may store instructions that, when executed by the processing structure, cause the processing structure to characterize the 3D object model in the surgical planning application as at least a portion of a no-drilling zone.

In the example device, the memory may store instructions that, when executed by the processing structure, cause the processing structure to characterize the 3D object model in the surgical planning application as at least a portion of a drilling zone.

In the example device, the 3D object model may contain a plurality of diameters including the outermost diameter.

### BRIEF DESCRIPTION OF THE DRAWINGS

For a detailed description of example embodiments, reference will now be made to the accompanying drawings in which:

FIG. 1 shows an anterior or front elevation view of right knee, with the patella removed;

FIG. 2 shows a posterior or back elevation view of the right knee;

FIG. 3 shows a view of the femur from below and looking into the intercondylar notch;

FIG. 4 shows a surgical system in accordance with at least some embodiments;

FIG. 5 shows portions of two 3D bone models corresponding respectively to the lower portion of a femur and the upper portion of a tibia;

FIG. 6 shows a 3D tunnel model in isolation;

FIG. 7 shows a 3D point cloud in a 3D coordinate system corresponding to the 3D tunnel model of FIG. 6;

FIG. 8 shows a 3D cylinder model being displayed in a 3D coordinate system in association with the 3D point cloud of FIG. 7;

FIG. 9 shows a conceptual drawing of a surgical site with various objects within the surgical site tracked, in accordance with at least some embodiments;

FIG. 10 is an example video display showing portions of a femur and having visible therein a bone fiducial, in accordance with at least some embodiments;

FIG. 11 shows a method in accordance with at least some embodiments; and

FIG. 12 shows a computer system in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

“Receiving . . . a . . . location” shall mean receiving data indicative of location on a bone within a coordinate space (e.g., a coordinate space of a view of an endoscope). Thus, example systems and methods may “receive . . . a revised-tunnel entry location” being data indicative of a proposed location of a tunnel entry point within a three-dimensional coordinate space. Other example systems and methods may “receive . . . a plurality of locations on a bone” being data indicative locations of an outer surface of a bone as part of registering a bone to a three-dimensional bone model.

An endoscope having “a single optical path” through an endoscope shall mean that the endoscope is not a stereoscopic endoscope having two distinct optical paths separated by an interocular distance at the light collecting end of the endoscope. The fact that an endoscope has two or more optical members (e.g., glass rods, optical fibers) forming a single optical path shall not obviate the status as a single optical path.

“Throughbore” shall mean an aperture or passageway through an underlying device. However, the term “throughbore” shall not be read to imply any method of creation. Thus, a throughbore may be created in any suitable way, such as drilling, boring, laser drilling, or casting.

“Counterbore” shall mean an aperture or passageway into an underlying device. In cases in which the counterbore intersects another aperture (e.g., a throughbore), the counterbore may thus define an internal shoulder. However, the term “counterbore” shall not be read to imply any method of creation. A counterbore may be created in any suitable way, such as drilling, boring, laser drilling, or casting.

“Processing structure” or “processor” shall mean a single processing device, processor, microprocessing device, microprocessor, computing device, computer, computer system or other device that, like these, can be instructed to and/or configured to conduct computational processing, or an arrangement of multiple processing devices, processors, microprocessing devices, microprocessors, computing devices, computers, computer systems and/or other devices that, like these, can be instructed to and/or configured to conduct computational processing.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to methods and systems for generating 3D models of existing bone tunnels for surgical planning.

The various examples were developed in the context of ACL repair, and thus the discussion below is based on the developmental context. However, the techniques are applicable to many types of ligament repair, such as medial collateral ligament repair, lateral collateral ligament repair, and posterior cruciate ligament repair. Moreover, the various example methods and systems can also be used for planning and placing anchors to reattach soft tissue, such as reattaching the labrum of the hip, the shoulder, or the meniscal root. Furthermore, the various example methods and systems can also be used for planning and navigation of instruments with respect to an anatomy. Thus, the description and developmental context shall not be read as a limitation of the applicability of the teachings. In order to orient the reader, the specification first turns a description of the knee.

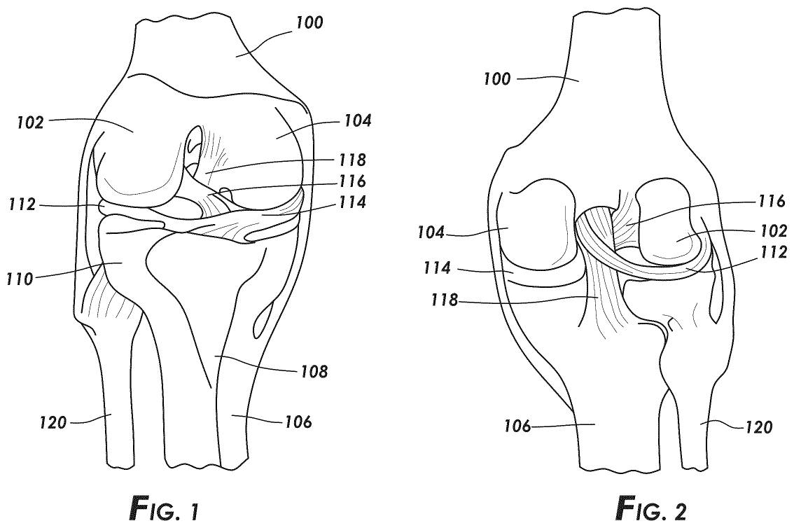

FIG. 1 shows an anterior or front elevation view of a right knee, with the patella removed. In particular, visible in FIG. 1 is lower portion of the femur 100 including the outer or lateral condyle 102 and the inner or medial condyle 104. The femur 100 and condyles 102 and 104 are in operational relationship to a tibia 106 including the tibial tuberosity 108 and Gerdy's tubercle 110. Disposed between the femoral condyles 102 and 104 and the tibia 106 are the lateral meniscus 112 and the medial meniscus 114. Several ligaments are also visible in the view of FIG. 1, such as the ACL 116 extending from the lateral side of femoral notch to the medial side of the tibia 106. Oppositely, the posterior cruciate ligament 118 extends from the medial side of the femoral notch to the tibia 106. Also visible is the fibula 120, and several additional ligaments that are not specifically numbered.

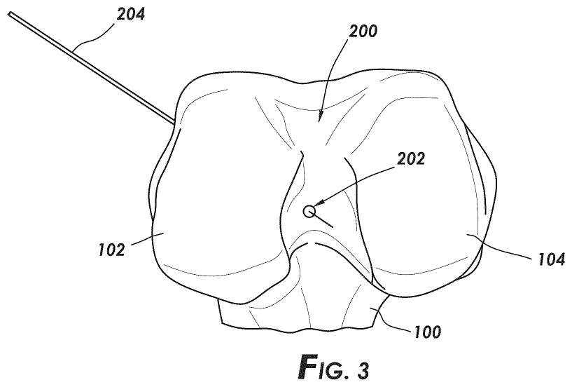

FIG. 2 shows a posterior or back elevation view of the right knee. In particular, visible in FIG. 2 is lower portion of the femur 100 including the lateral condyle 102 and the medial condyle 104. The femur 100 and femoral condyles 102 and 104 again are in operational relationship to the tibia 106, and disposed between the femoral condyles 102 and 104 and the tibia 106 are the lateral meniscus 112 and the medial meniscus 114. FIG. 2 further shows the ACL 116 extending from the lateral side of femoral notch to the medial side of the tibia 106, though the attachment point to the tibia 106 is not visible. The posterior cruciate ligament 118 extends from medial side of the femoral notch to the tibia 106, though the attachment point to the femur 100 not visible. Again, several additional ligaments are shown that are not specifically numbered.

The most frequent ACL injury is a complete tear of the ligament. Treatment involves reconstruction of the ACL by placement of a substitute graft (e.g., autograft from either the patellar tendon, quad tendon, or the hamstring tendons). The graft is placed into tunnels prepared within the femur 100 and the tibia 106. The current standard of care for ACL repair is to locate the tunnels such that the tunnel entry point for the graft is at the anatomical attachment location of the native ACL. Such tunnel placement at the attachment location of the native ACL attempts to recreate original knee kinematics. In arthroscopic surgery, the location of the tunnel through the tibia 106 is relatively easy to reach, particularly when the knee is bent or in flexion. However, the tunnel through the femur 100 resides within the intercondylar notch. Depending upon the physical size of the patient and the surgeon's selection for location of the port through the skin, and through which the various instruments are inserted into the knee, it may be difficult to reach the attachment location of the native ACL to the femur 100.

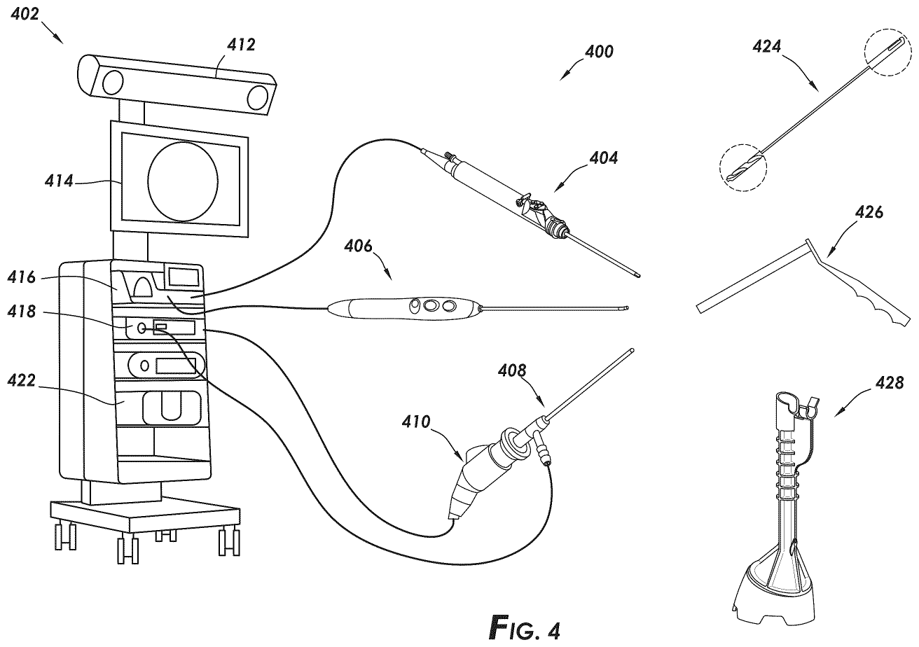

FIG. 3 shows a view of the femur from below and looking into the intercondylar notch. In particular, visible in FIG. 3 are the lateral condyle 102 and the medial condyle 104. Defined between the femoral condyles 102 and 104 is the femoral notch 200. The femoral tunnel may define an inside aperture 202 within the femoral notch 200, the inside aperture 202 closer to the lateral condyle 102 and displaced into the posterior portion of the femoral notch 200. The femoral tunnel extends through the femur 100 and forms an outside aperture on the outside or lateral surface of the femur 100 (the outside aperture not visible in FIG. 3). FIG. 3 shows an example drill wire 204 that may be used to create an initial tunnel or pilot hole. Once the surgeon verifies that the pilot hole is closely aligned with a planned-tunnel path, the femoral tunnel is created by boring or reaming with another instrument (e.g., a reamer) that may use the drill wire 204 as a guide. In some cases, a socket or counter-bore is created on the intercondylar notch side to accommodate the width of the graft that extends into the bone, and that counterbore may also be created using another instrument (e.g., reamer) that may use the drill wire 204 as a guide.

Drilling of a tunnel may take place from either direction. Considering the femoral tunnel again as an example, the tunnel may be drilled from the outside or lateral portion of the femur 100 toward and into the femoral notch 200, which is referred to as an “outside-in” procedure. Oppositely, the example femoral tunnel may be drilled from the inside of the femoral notch 200 toward and to the lateral portion of the femur 100, which is referred as an “inside-out” procedure. The various examples discussed below are equally applicable to outside-in or inside-out procedures. Outside-in procedures may additionally use a device which holds the drill wire on the outside portion, and physically shows the expected tunnel location of the inside aperture within the knee. However, the device for the outside-in procedure is difficult to use in arthroscopic procedures, and thus many arthroscopic repairs use the inside-out procedure. The specification now turns to an example surgical system.

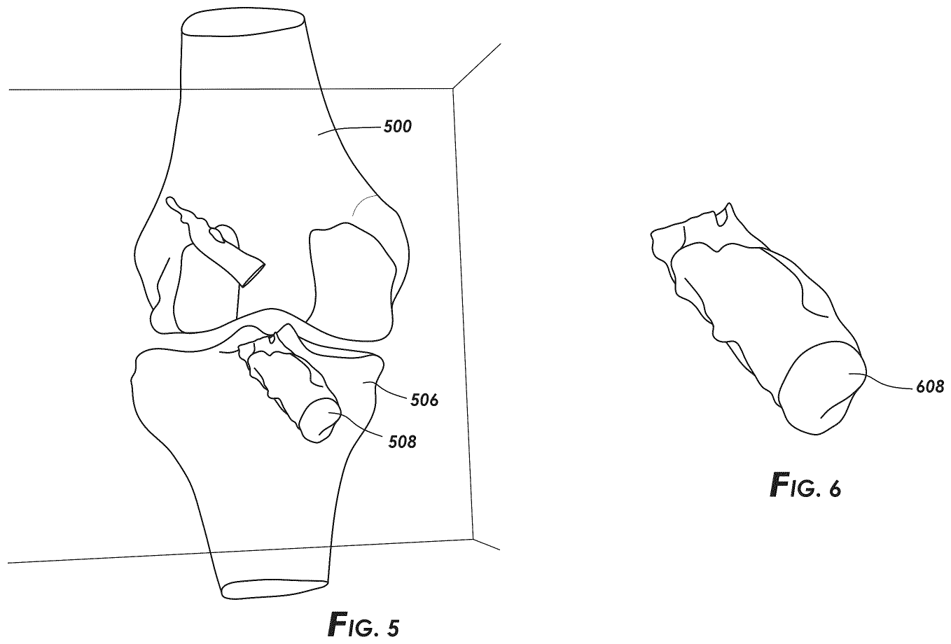

FIG. 4 shows a surgical system (not to scale) in accordance with at least some embodiments. In particular, the example surgical system 400 comprises a tower or device cart 402, an example mechanical resection instrument 404, an example plasma-based ablation instrument (hereafter just ablation instrument 406), and an endoscope in the example form of an arthroscope 408 and attached camera head 410. The endoscope 408 defines a light connection or light post 420 to which light is provided, and the light is routed internally within the endoscope 408 to illuminate a surgical field at the distal end of the endoscope 408. The device cart 402 may comprise a camera 412 (illustratively shown as a stereoscopic camera), a display device 414, a resection controller 416, and a camera control unit (CCU) together with an endoscopic light source and video controller 418. In example cases the CCU and video controller 418 provides light to the light post 420 of the arthroscope 408, displays images received from the camera head 410. In example cases, the CCU and video controller 418 also implements various additional aspects, such as calibration of the arthroscope and camera head, displaying planned-tunnel paths on the display device 414, receiving revised-tunnel entry locations, calculating revised-tunnel paths, and calculating and displaying various parameters that show the relationship between the revised-tunnel path and the planned-tunnel path. Thus, the CCU and video controller is hereafter referred to as surgical controller 418. In other cases, however, the CCU and video controller may be a separate and distinct system from the controller that handles aspects of intraoperative changes, yet the separate devices would nevertheless be operationally coupled.

The example device cart 402 further includes a pump controller 422 (e.g., single or dual peristaltic pump). Fluidic connections of the mechanical resection instrument 404 and ablation instrument 406 are not shown so as not to unduly complicate the figure. Similarly, fluidic connections between the pump controller 422 and the patient are not shown so as not to unduly complicate the figure. In the example system, both the mechanical resection instrument 404 and the ablation instrument 406 are coupled to the resection controller 416 being a dual-function controller. In other cases, however, there may be a mechanical resection controller separate and distinct from an ablation controller. The example devices and controllers associated with the device cart 402 are merely examples, and other examples include vacuum pumps, patient-positioning systems, robotic arms holding various instruments, ultrasonic cutting devices and related controllers, patient-positioning controllers, and robotic surgical systems.

FIG. 4 further shows additional instruments that may be present during an example ACL repair. In particular, FIG. 4 shows an example guide wire or drill wire 424 and an aimer 426. The drill wire 424 may be used to create an initial or pilot tunnel through the bone. In some cases, the diameter of the drill wire may be about 2.4 millimeters (mm), but larger and smaller diameters for the drill wire 424 may be used. The example drill wire 424 is shown with magnified portions on each end, one to show the cutting elements on the distal end of the drill wire 242, and another magnified portion to show a connector for coupling to chuck of a drill. Once the surgeon drills the pilot tunnel, the surgeon and/or the surgical controller 418 (discussed more below) may then assess whether the pilot tunnel matches or closes matches the planned-tunnel path. If the pilot tunnel is deemed sufficient, then the drill wire 424 may be used as a guide for creating the full-diameter throughbore for the tunnel, and possibly also for creating a counterbore associated with intercondylar notch to accommodate the graft. While in some cases the drill wire alone may be used when creating the pilot tunnel, in yet still other cases the surgeon may use the aimer 426 to help guide and place the drill wire 424 at the designed tunnel-entry location.

FIG. 4 also shows that the example system may comprise a calibration assembly 428. As explained in further detail in PCT Publication No. WO/2023/034194 to Quist et al. (“Quist”), the contents of which are incorporated by reference herein, the calibration assembly 428 may be used to detect optical distortion in images received by the surgical controller 418 through the arthroscope 408 and attached camera head 410. Additional tools and instruments will be present, such as a drill for drilling with the drill wire 424, various reamers for creating the throughbore and counterbore aspects of the tunnel, and various tools for suturing and anchoring the graft in place. These additional tools and instruments are not shown so as not to further complicate the figure.

The specification now turns to a workflow for an example revision ACL reconstruction. The workflow may be conceptually divided into preoperative planning and intraoperative repair. The intraoperative repair workflow may be further conceptually divided into optical system calibration, model registration, intraoperative tunnel-path planning, intraoperative tunnel creation, and intraoperative tunnel placement analysis. Each will be addressed in turn.

### Planning

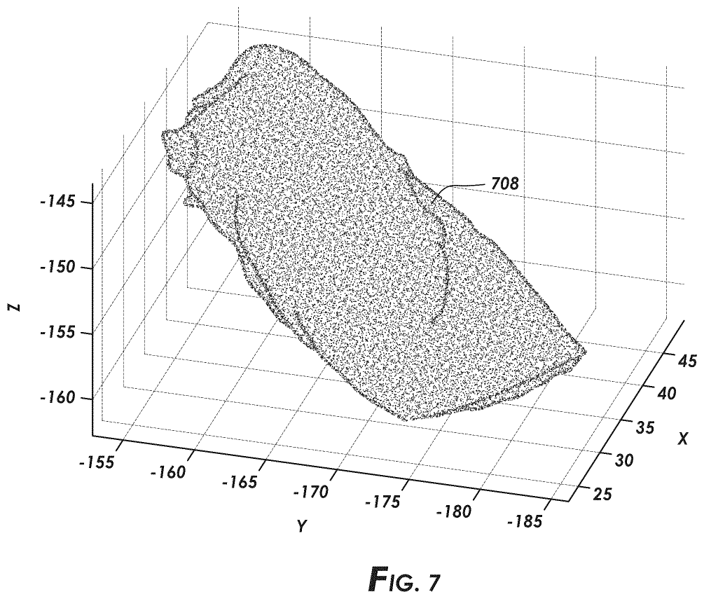

In accordance with various examples, an ACL repair such as a revision ACL reconstruction starts with imaging (e.g., X-ray imaging, computed tomography (CT), magnetic resonance imaging (MRI)) of the knee of the patient, including the relevant anatomy like the lower portion of the femur, the upper portion of the tibia, and the articular cartilage. Imaging in this context may involve capture of multiple cross-sectional images or slices. The discussion that follows assumes MRI imaging, but again many different types of imaging may be used. The MRI imaging can be segmented from the image slices such that a volumetric model or three-dimensional model of the anatomy is created. Any suitable currently available, or after developed, segmentation technology may be used to create the three-dimensional model. More specifically to the example of revision ACL reconstruction and specifically selecting a tunnel path through the tibia, a three-dimensional (3D) bone model of the upper portion of the tibia, including the femoral condyles, is created. FIG. 5 shows portions of two 3D bone models 500, 506 corresponding respectively to the lower portion of the femur 100 and the upper portion of the tibia 106 shown in FIGS. 1 and 2.

Particularly relevant to revision ACL reconstruction, the 3D bone model 506 of the upper portion of the tibia may incorporate 3D features defining one or more pre-existing bone tunnels 508 in the tibia. Each of the pre-existing bone tunnel(s) 508 may be represented and manipulated as a respective 3D tunnel model 608, as shown in FIG. 6. In an example, tunnel features in each of multiple cross-sectional images of the upper portion of the tibia 106 may be segmented and the 3D tunnel model 608 generated based on the segmented tunnel features. In an example, the tunnel features may be those features in the 3D bone model 506 that represent a void within the bone. This, as contrasted with those features in the 3D bone model 506 that represent bone itself. In an example, a mask representing the tunnel features may be generated for each of the multiple cross-sectional images. The masks in the cross-sectional images may be together processed to generate the 3D tunnel model 608. Such a 3D tunnel model 608, like the 3D bone model 506, may be represented in a 3D file and viewed and manipulated as such on a display device, such as display device 414 (FIG. 4). The 3D tunnel model 608 may be viewed and/or manipulated in isolation as shown in FIG. 6 and/or in association with the 3D bone model 506. The cross-sectional images on which the 3D tunnel model 608 is based are typically very high-resolution images carrying detailed image content representing fine, dimensionally-accurate distinctions between bone and voids. Consequently, the 3D tunnel model 608 may also embody fine, dimensionally-accurate detail for its surfaces and contours.

Due to a number of factors, such as bone in-growth that may have occurred since a tunnel was initially drilled for the primary ACL reconstruction, a 3D tunnel model 608 of the tunnel generated as a result of processing the cross-sectional images of the bone, or otherwise generated, may be difficult for a human observer to characterize. For example, the 3D tunnel model 608 may be difficult to characterize as a particular geometric shape, such as a cylinder, having a well-defined orientation, a well-defined diameter, and a well-defined length. If the 3D tunnel model 608 is “lumpy” due to the tunnel the 3D tunnel model 608 represents being itself lumpy, it can be difficult for a human observer such as a surgeon to discern a single particular orientation, a single particular diameter, and a single particular length.

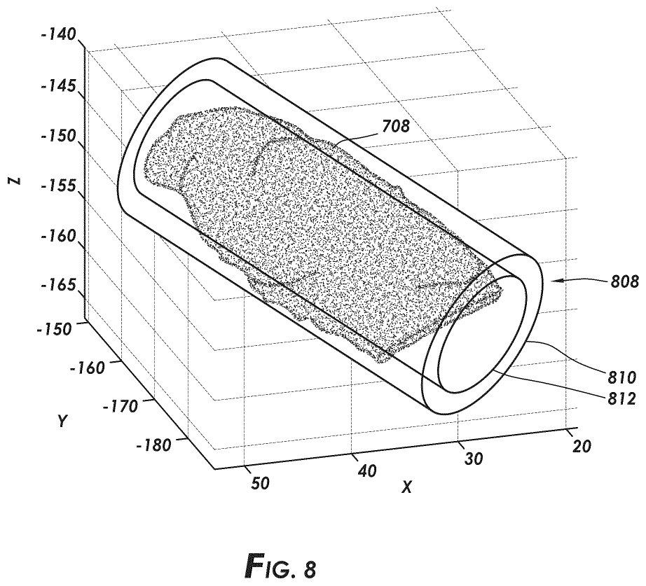

In order to cause data embodied in the 3D tunnel model 608 to be useful for downstream automatic manipulation, a point cloud may be automatically generated based on the 3D tunnel model and/or based more directly on the cross-sectional images. A point cloud 708 corresponding to 3D tunnel model 608 is shown in FIG. 7 within a 3D coordinate system having X, Y, and Z axes. In FIG. 7, each of the X-Y, X-Z, and Y-Z planes of the 3D coordinate system are depicted using respective grid lines. The grid lines provide visual context for the point cloud 708 within the 3D coordinate system and visual indications as to the dimensions of point cloud 708. In this example, point cloud generation initiates with a conversion of a slice boundary to a discretized set of points along the contour of a labeled layer. Interpolation of points between slices is conducted as a linear interpolation between nearest neighbor points occurring on the labeled boundary. In this example, each of the points of the point cloud has an (x,y,z) position coordinate in the 3D coordinate system.

The 3D tunnel model 608 and the corresponding point cloud 708 may each generally be regarded by a person to represent a somewhat-cylindrical shape, or to represent another shape of enclosed N-sided polygon cross-section generally uniform along its length. The somewhat-cylindrical or other shape may be regarded as oriented to extend from a location at the back-top-left of the 3D coordinate system to another location at the front-bottom-right. However, because of the significant surface variation along its extents in each of the dimensions, and because of the challenges presented to a user of manipulating the displayed 3D tunnel model 608 or its point cloud 708, it may be difficult, time-consuming, and at risk of significant error for the person to manually, through visual inspection, specify the parameters of a shape. The person may not generally regard the point cloud 708 as having even a somewhat-cylindrical or other somewhat uniform cross-sectional shape. For example, it may be difficult, time-consuming, and prone to error for the person to visually inspect the 3D tunnel model 608 or the point cloud 708 to specify an exact long axis, an exact outer periphery (or, in the case of a cylinder, an exact outer diameter), and exact endpoints of an object such as a cylinder.

In order to automatically and accurately characterize the point cloud 708 as a uniform-cross sectional object such as a cylinder, the points of the point cloud may be processed to determine at least a first principal axis of the point cloud in the 3D coordinate system. In this example, the first principal axis may be regarded as the longest axis through the point cloud as determined using a principal component analysis (PCA) process such as a single value decomposition (SVD) process That is, the first principal axis may be regarded as the line along which the most variation in the position of the points of the point cloud occurs, and thus may be indicative of the central axis of a notional cylinder that is at least partially represented by the point cloud, or the central axis of another notional object of uniform cross-sectional shape. It will be appreciated that the first principal axis may be considered the Eigenvector of the point cloud, oriented at a particular angle in the 3D coordinate system.

The points of the point cloud may, in addition, be processed to determine a second principal component axis, which may be regarded as a line perpendicular to the first principal component axis along which the second most variation in the position of the points of the point cloud occurs. The points of the point cloud may also be processed to determine a third principal axis, which may be regarded as the line perpendicular to the first and second principal component axes and, incidentally, along which the third most variation in the position of the points of the point cloud occurs.

Based on the first principal axis, endpoints of the point cloud are determined. The endpoints may be regarded, respectively, as the proximal and distal points on the notional cylinder or other object of uniform cross-section that, as described above, has a central axis that is coincidental to the first principal axis.

With the endpoints having been determined, each of the 3D points of the point cloud may be projected onto a two-dimensional (2D) plane that is perpendicular to the first principal axis. The 3D points, once projected, may be regarded as 2D projected points. In an example, prior to the projecting of the 3D points onto the 2D plane, each point of the point cloud is rotated in 3D space to align the first principal axis of the point cloud with a z axis of the coordinate system. In an example, each point of the point cloud may be rotated to cause the first principal axis of the point cloud to align with the origin of the 3D coordinate system, thereby to be coincident with the z axis. In such examples, after the rotating, the 2D plane that is perpendicular to the first principle axis is an x-y plane in the coordinate system.

The following is described for ease of understanding with reference to a 3D object model to be generated as being a 3D cylinder model in particular. It will be appreciated that the following will also be applicable to other cross-sectional shapes that may be characterized as being non-circular, but like circles as having enclosed N-sided polygon cross-sections. Outermost and innermost peripheries of such enclosed N-sided polygons are referred to, in the case of the N-sided polygons being circles, as outermost and innermost diameters of first, second, and third circles.

With the projected points having been projected onto the 2D plane, a first circle in the 2D plane centered on the first principal axis and enclosing the projected points is determined. The first circle may be regarded as an outermost diameter of the notional cylinder having the determined endpoints and its central axis coincident to the first principal axis. In an example, the first circle is a best-fit circle. In another example, the first circle is larger in diameter by a first tolerance amount than a best-fit circle. The tolerance amount may represent a margin of error available to, or provided by, the surgeon when using the outermost diameter as representative of a “no-fly” zone. In this description, a no-fly zone is a region to be avoided when drilling/reaming intraoperatively. By providing a margin of error, a surgeon may avoid drilling into or close enough to the existing tunnel that a too-thin wall of bone between the existing tunnel and a new tunnel is susceptible to collapse. Such collapse may be at risk of occurring during a surgical operation or afterwards. A margin of error in the outermost diameter may not be as important to a surgeon when planning to over-drill or bore out the existing bone tunnel to prepare it for re-use during revision ACL reconstruction.

In an example, a second circle in the 2D plane centered on the first principle axis and enclosed by the projected points may be determined. This determination may be used to provide an innermost diameter of the existing tunnel. The innermost diameter may be considered to represent, for example, an outer diameter of a rigid drill wire that could be passed through the existing bone tunnel centered on the first principal axis without being blocked by any bone in-growth that may have occurred around the periphery of the bone tunnel since it was originally drilled. In an example, the second circle is a best-fit circle. In another example, the second circle is smaller in diameter by a second tolerance amount than a best-fit circle. The tolerance amount may represent a margin of error available to, or provided by, the surgeon.

In various examples, the first circle may be a best-fit circle and a third circle may be determined that is coaxial with the first circle but is greater in diameter than the first circle by a tolerance amount representative of a margin of error. Similarly, in various examples, the second circle may be a best-fit circle and a fourth circle may be determined that is coaxial with the first and second circles but is smaller in diameter than the second circle by a tolerance amount representative of a margin of error. Variations are possible.

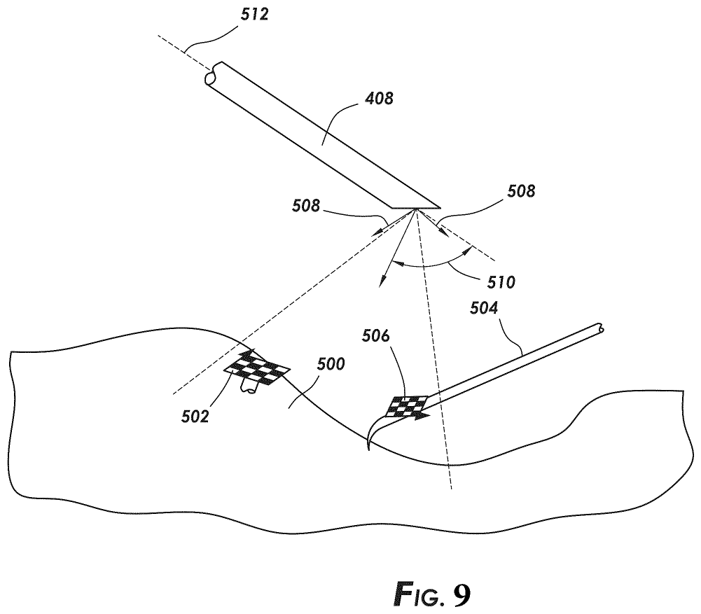

With the characteristics of the outermost diameter, the first principal axis, the endpoints, and optionally the innermost diameter, having been determined, a 3D cylinder model is generated based on the characteristics. For example, the 3D cylinder model may represent a cylinder with a long axis corresponding to the first principal axis, a diameter corresponding to the outermost diameter, and a length corresponding to the span between the endpoints. The 3D cylinder model may thereafter be provided to a surgical planning application for downstream use during planning for the revision ACL reconstruction. The 3D cylinder model may be displayed in association with the 3D tunnel model and/or the 3D bone model, for example as a translucent overlay. FIG. 8 shows the 3D point cloud 708, and a 3D cylinder model 808 generated based on the 3D point cloud 708 being displayed. In FIG. 8, the outermost diameter 810 of the 3D cylinder model 808 based on the first circle and the innermost diameter 812 of the 3D cylinder model 808 based on the second circle are both displayed as contrasting translucent overlays so that they may be distinguished visually from each other and each from the point cloud 708 by a user.

The 3D cylinder model 808 may be characterized in the surgical planning application as a no-drilling zone, such that a surgeon is to avoid planning for and drilling tunnels that would intersect the 3D cylinder model 808 and, accordingly, the existing bone tunnel. This may be useful should the surgeon determine that it is preferable to avoid creating tunnels during revision ACL reconstruction with tunnel walls that may be weakened by their proximity or coincidence with an existing bone tunnel. Alternatively, the 3D cylinder model may be characterized in the surgical planning application as at least a portion of a drilling zone, such that a surgeon is to plan for and drill tunnels that align with the 3D tunnel model and, accordingly, the existing bone tunnel. This may be useful for boring out the existing bone tunnel should the surgeon determine that it was, and is still, suitably located and oriented, as well as suitably structurally healthy/sound, for use in receiving an allograft or autograft bone plug and/or hardware, during a revision ACL reconstruction. It will be appreciated that a surgeon may consider it useful to minimize healthy bone removal when providing a consistent surface for bone-to-bone contact with a bone plug. Furthermore, the surgeon may use the 3D cylinder model 808 to select bone plug diameter(s) and/or to guide a navigated instrument such as a reamer.

In various examples, additional variations in the characteristics of the 3D cylinder model may be provided. In one example, a variable-radius cylinder may be generated. In such an example, a first percentage of the length of the cylinder along the first principal axis (i.e., the long axis) may be best-fit, with the remainder of the length of the cylinder along the same first principal axis being provided with a tolerance amount. In accordance with such an example, a first 2D plane upon which a subset of the point cloud points can be projected may be considered located at a position along the first principal axis corresponding to the first percentage. A second 2D plane upon which the remainder of the point cloud points can be projected may be considered located at an endpoint along the first principal axis. Various options are available for determining best fit circles along the principal axis and registering step changes between such best fit circles.

Using the 3D bone model and the 3D cylinder model, an operative plan is created that comprises choosing a planned-tunnel path through the tibia, including locations of the apertures of the bone that define the ends of the new tunnel.

It will be appreciated that the processes and systems described herein with respect to the tibia are also useful for determining 3D cylinder models representative of respective existing tunnels in a femur. For an example inside-out repair, the aperture within the femoral notch is the entry location for the drilling, and the aperture on the lateral surface of the femur is the exit location. For an outside-in repair, the entry and exit locations for drilling are swapped. Still assuming an inside-out repair, the entry location may be selected to be the same as, or close to, the attachment location of the native ACL to the femur within the femoral notch. In some cases, selecting the entry location within the femoral notch may involve use of a Bernard & Hertel Quadrant or grid placed on a fluoroscopic image, or placing the Bernard & Hertel Quadrant on a simulated fluoroscopic image created from the three-dimensional bone model. Based on use of the Bernard & Hertel Quadrant, an entry location for the tunnel is selected. For an inside-out repair, selection of the exit location is less restrictive, not only because the portion of the tunnel proximate to the exit location is used for placement of the anchor for the graft, but also because the exit location is in approximately centered in the femur (considered anteriorly to posteriorly), and thus issues of bone wall thickness at the exit location are of less concern. In some cases, a three-dimensional bone model of the proximal end of the tibia is also created, and the surgeon may likewise choose planned-tunnel path(s) through the tibia.

The results of the planning for a revision ACL reconstruction may comprise: a 3D bone model of the distal end of the femur; a 3D bone model for a proximal end of the tibia; a 3D cylinder model for each existing tunnel in the distal end of the femur; a 3D cylinder model for each existing tunnel in the proximal end of the tibia; an entry location and exit location through the femur and thus a planned-tunnel path for the femur, whether it be coincident with an existing tunnel or planned to avoid the existing tunnel; and an entry location and exit location through the tibia and thus a planned-tunnel path through the tibia, whether it be coincident with an existing tunnel or planned to avoid the existing tunnel. Other surgical parameters may also be selected during the planning, such as tunnel throughbore diameters, tunnel counterbore diameters and depth, desired post-repair flexion, and the like, but those additional surgical parameters are omitted from this description so as not to unduly complication the specification.

### Intraoperative Repair

The specification now turns to intraoperative aspects. The intraoperative aspects include steps and procedures for setting up the surgical system to perform the various repairs. It is noted, however, that some of the intraoperative aspects (e.g., optical system calibration), may take place before any ports or incisions are made through the patient's skin, and in fact before the patient is wheeled into the surgical room. Nevertheless, such steps and procedures may be considered intraoperative as they take place in the surgical setting and with the surgical equipment and instruments used to perform the actual repair.

The example revision ACL reconstruction is conducted arthroscopically and is computer-assisted in the sense the surgical controller 418 is used for arthroscopic navigation within the surgical site. More particularly, in example systems the surgical controller 418 provides computer-assistance during the ligament repair by tracking location of various objects within the surgical site, such as the location of the bone within the three-dimensional coordinate space of the view of the arthroscope, and location of the various instruments (e.g., the drill wire 424, the aimer 426) within the three-dimensional coordinate space of the view of the arthroscope. The specification turns to brief description of such tracking techniques.

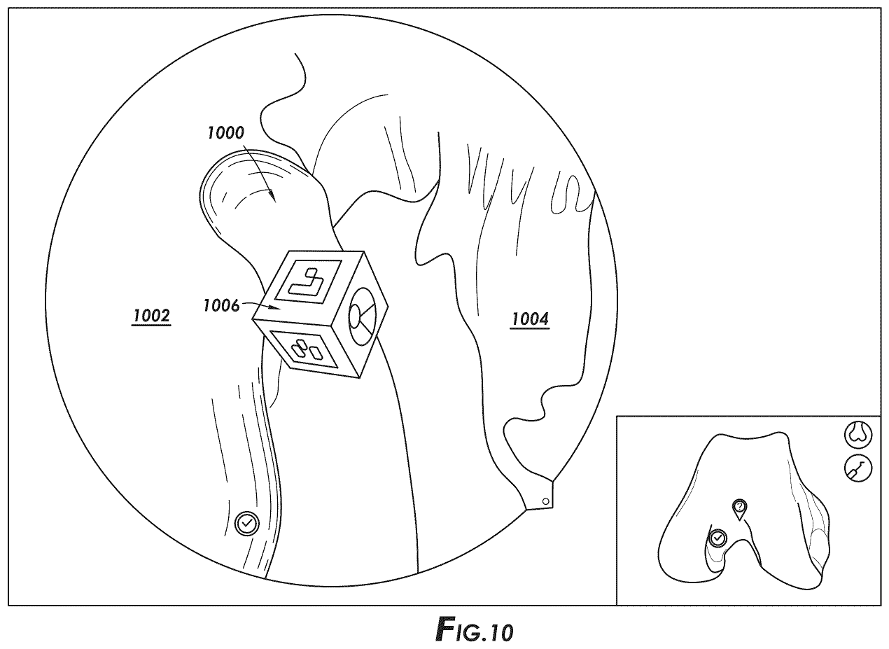

FIG. 9 shows a conceptual drawing of a surgical site with various objects within the surgical site. In particular, visible in FIG. 9 is a distal end of the arthroscope 408, a portion of a bone 500 (e.g., femur), a bone fiducial 502 within the surgical site, a touch probe 504, and a probe fiducial 506. Each is addressed in turn.

The distal end of the arthroscope 408 is designed and constructed to illuminate the surgical site with visible light received by way of the light post 420 (FIG. 4). In the example of FIG. 9, the illumination is illustrated by arrows 508. The illumination provided to the surgical site is reflected by various objects and tissues within the surgical site, and the reflected light that returns to the distal end enters the arthroscope 408, propagates along an optical channel within the arthroscope 408, and is eventually incident upon a capture array within the camera head 410 (FIG. 4). The images detected by the capture array within the camera head 410 are sent electronically to the surgical controller 418 (FIG. 4) and displayed on the display device 414 (FIG. 4). In accordance with example systems, the arthroscope 408 has a single optical path through the arthroscope for capturing images of the surgical site, notwithstanding that the single optical path may be constructed of two or more optical members (e.g., glass rods, optical fibers). That is to say, in example systems and methods the computer-assisted navigation provided by the arthroscope 408, camera head 410, and surgical controller 418 is provided with the arthroscope 408 that is not a stereoscopic endoscope having two distinct optical paths separated by an interocular distance at the distal end endoscope.

During a surgical procedure, a surgeon selects an arthroscope with a viewing direction beneficial for the planned surgical procedure. Viewing direction refers to a line residing at the center of an angle subtended by the outside edges or peripheral edges of the view of an endoscope. The viewing direction for some arthroscopes is aligned with the longitudinal central axis of the arthroscope, and such arthroscopes are referred to as “zero degree” arthroscopes (e.g., the angle between the viewing direction and the longitudinal central axis of the arthroscope is zero degrees). The viewing direction of other arthroscopes forms a non-zero angle with the longitudinal central axis of the arthroscope. For example, for a 30° arthroscope the viewing direction forms a 30° angle to the longitudinal central axis of the arthroscope, the angle measured as an obtuse angle beyond the distal end of the arthroscope. In many cases for ACL repair, the surgeon selects a 30° arthroscope or a 45° arthroscope based on location the port created through the skin of the patient. In the example of FIG. 9, the view angle 510 of the arthroscope 408 forms a non-zero angle to the longitudinal central axis 512 of the arthroscope 408.

Still referring to FIG. 9, within the view of the arthroscope 408 is a portion of the bone 500, along with the bone fiducial 502, the touch probe 504, and the probe fiducial 506. The bone fiducial 502 is shown as a planar element having a pattern disposed thereon, though other shapes for the bone fiducial 502 may be used (e.g., a square block with a pattern on each face of the block). The bone fiducial 502 may be attached to the bone 500 in any suitable form (e.g., a fastener, such as a screw). The pattern of the bone fiducial is designed to provide information regarding the orientation of the bone fiducial 502 in the three-dimensional coordinate space of the view of the arthroscope 408. More particularly, the pattern is selected such that the orientation of the bone fiducial 502, and thus the orientation of the underlying bone 500, may be determined from images captured by the arthroscope 408 and attached camera head 410 (FIG. 4).

The probe fiducial 506 is shown as a planar element attached to the touch probe 504. The touch probe 504 may be used, as discussed more below, to “paint” the surface of the bone 500 as part of the registration of the bone 500 to the three-dimensional bone model, and the touch probe 504 may also be used to indicate revised-tunnel entry locations in the case of intraoperative changes to the tunnel paths. The probe fiducial 506 is shown as a planar element having a pattern disposed thereon, though other shapes for the probe fiducial 506 may be used (e.g., a square block surrounding the touch probe 504 with a pattern on each face of the block). The pattern of the probe fiducial 506 is designed to provide information regarding the orientation of the probe fiducial 506 in the three-dimensional coordinate space of the view of the arthroscope 408. More particularly, the pattern is selected such that the orientation of the probe fiducial 506, and thus the location of the tip of the touch probe 504, may be determined from images captured by the arthroscope 408 and attached camera head 410 (FIG. 4).

Other instruments within the view of the arthroscope 408 may also have fiducials, such as the drill wire 424 (FIG. 4) and aimer 426 (FIG. 4), but the additional instruments are not shown so as not unduly complicate the figure. Moreover, in addition to or in place of tracking location based on the view through the arthroscope 408, the location of the distal end of one or more of the instruments may be tracked by other methods and systems. For example, for devices that rigidly extend out of the surgical site (e.g., the aimer 426 (FIG. 4)), the location may be tracked by an optical array coupled to the aimer and viewed through the camera 412 (FIG. 4), such as a stereoscopic camera. As another example, the shape of an instrument such as a touch probe may be itself automatically recognizable, for example as a result of programming and/or machine-learning enabling recognition of the instrument in arthroscopic images, such that the position and orientation of the instrument may be gleaned automatically without it having to itself carry a fiducial. The location within the three-dimensional coordinate space of the camera 412 is then transformed into the three-dimensional coordinate space of the view of the example arthroscope to determine location of the distal end within the surgical site.

The images captured by the arthroscope 408 and attached camera head 410 are subject to optical distortion in many forms. For example, the visual field between distal end of the arthroscope 408 and the bone 500 within the surgical site is filled with fluid, such as bodily fluids and saline used to distend the joint. Many arthroscopes have one or more lenses at the distal end that widen the field of view, and creating wider field of view causes a “fish eye” effect in the captured images. Further, the optical elements within the arthroscope (e.g., rod lenses) may have optical aberrations inherent to the manufacturing and/or assembly process. Further still, the camera head 410 may have various optical elements for focusing the images receives onto the capture array, and the various optical elements may have aberrations inherent to the manufacturing and/or assembly process. As explained in further detail in Quist, in example systems and methods, prior to use within each surgical procedure, the endoscopic optical system is calibrated to account for the various optical distortions. In an example calibration procedure, the example surgical controller 418 creates a characterization function that characterizes optical distortion between the calibration target and the capture array within the camera head 410. The characterization function may include a calibration for determining orientation of fiducial markers visible within the surgical site (e.g., bone fiducial 502, probe fiducial 506) by way of the arthroscope 408 and attached camera head 410.

The next example step in the intraoperative procedure is the registration of the bone model(s). That is, during the planning stage, imaging (e.g., MRI) of the knee takes place, including the relevant anatomy like the lower portion of the femur, the upper portion of the tibia, and the articular cartilage. The imaging can be segmented such that a volumetric model or three-dimensional model of the anatomy is created from cross-sectional images captured during the imaging. More specifically to the example of ACL repair, and specifically selecting a tunnel path through the femur, a three-dimensional bone model of the lower portion of the femur is created during the planning.

During the intraoperative repair, the three-dimensional bone models and the cross-sectional images are provided to the surgical controller 418. Again using the example of ACL repair, and specifically computer-assisted navigation for tunnel paths through the femur, the three-dimensional bone model of the lower portion of the femur is provided to the surgical controller 418. Thus, the surgical controller 418 receives the three-dimensional bone model, and assuming the arthroscope 408 is inserted into the knee by way of a port through the patient's skin, the surgical controller 418 also receives video images of the femur. In accordance with example methods, the surgical controller 418 may be provided, and thus may receive, the cross-sectional images captured during the imaging from the planning stage.

In order to relate the three-dimensional bone model to the images received by way of the arthroscope 408 and camera head 410, the surgical controller 418 registers the three-dimensional bone model to the images of the femur received by way of the arthroscope 408 and camera head 410.

By way of the 3D bone model of the femur being registered to the images containing the femur, and the 3D bone model of the tibia being registered to the images containing the tibia, the 3D cylinder model(s) of existing bone tunnels in the femur and in the tibia may also themselves be regarded as registered to the images containing, respectively, the femur and the tibia.

In accordance with example methods, a fiducial marker or bone fiducial (e.g., bone fiducial 502 of FIG. 9) is attached to the femur. The bone fiducial placement is such that the bone fiducial is within the field of view of the arthroscope 408, but in a location spaced apart from the expected tunnel entry/exit point through the lateral condyle. More particularly, in example cases the bone fiducial is placed within the intercondylar notch superior to or above the expected location of the tunnel through lateral condyle.

FIG. 10 is an example video display showing portions of a femur and a bone fiducial. The display may be shown, for example, on the display device 414 (FIG. 4) associated with the device cart 402 (FIG. 4), or any other suitable location. In particular, visible in FIG. 10 is a femoral notch or intercondylar notch 1000, a portion of the lateral condyle 1002, a portion the medial condyle 1004, and an example bone fiducial 1006. The bone fiducial 1006 is a fiducial comprising a cube member. Of the six outer faces of the cube member, the bottom face is associated with an attachment feature (e.g., a screw). The bottom face will be close to or will abut the bone when the bone fiducial 1006 is secured in place, and thus will not be visible in the view of the arthroscope 408 (FIG. 4). The outer face opposite the bottom face includes a placement feature used to hold the bone fiducial 1006 prior to placement, and to attach the bone fiducial 1006 to the underlying bone. Of the remaining four outer faces of the cube member (only two of the remaining faces are visible), each of the four outer faces has a machine-readable pattern thereon, and in some cases each machine-readable pattern is unique. Once placed, the bone fiducial 1006 represents a fixed location on the outer surface of the bone in the view of the arthroscope 408, even as the position of the arthroscope 408 is moved and changed relative to the bone fiducial 1006. Initially, the location of the bone fiducial 1006 with respect to the three-dimensional bone model is not known to the surgical controller 418, hence the need for the registration of the three-dimensional bone model.

In order to relate or register the bone visible in the video images to the three-dimensional bone model, and accordingly to relate or register the bone visible in the video images to respective 3D cylinder models, the surgical controller 418 (FIG. 4) is provided and thus receives a plurality of locations of an outer surface of the bone. For example, the surgeon may touch a plurality of locations using the touch probe 504 (FIG. 9). As previously discussed, the touch probe 504 comprises a probe fiducial 506 (FIG. 9) visible in the video images captured by the arthroscope 408 (FIG. 4) and camera head 410 (FIG. 4). The physical relationship between the distal end of the touch probe 504 and the probe fiducial 506 is known by the surgical controller 418, and thus as the surgeon touches each of the plurality of locations on the outer surface of the bone, the surgical controller 418 gains an additional “known” locations of the outer surface of the bone relative to the bone fiducial 1006. Given that the touch probe 504 is a relatively inflexible instrument, in other examples the tracking of the touch probe 504 may be by optical tracking of an optically-reflective array outside the surgical site (e.g., tracking by the camera 412 (FIG. 4)) yet attached to the portion of the touch probe 504 inside the surgical site.

In some cases, particularly when portions of the outer surface of the bone are exposed to view, receiving the plurality of locations of the outer surface of the bone may involve the surgeon “painting” the outer surface of the bone. “Painting” is a term of art that does not involve application of color or pigment, but instead implies motion of the touch probe 504 when the distal end of the touch probe 504 is touching bone.

Further details of registering a three-dimensional bone model to images of a bone received by way of the arthroscope 408 and camera head 410 will not be described further herein. However, a number of systems, methods and procedures for conducting such a registration are described in Quist. It will be appreciated, however, that due to the correlation between a 3D bone model as described herein and a 3D cylinder model representing a tunnel through the bone, registering the images of the bone(s) and the 3D bone model(s) is to also register the images of the bone and the 3D cylinder model(s).

Using the three-dimensional bone model and the 3D cylinder model an operative plan may be created that comprises a planned-tunnel path through the bone, including locations of the apertures into the bone that define the ends of the tunnel. In some cases, however, the surgeon may elect not to use planned-tunnel path, and thus elect not use the planned entry location, exit location, or both. Such an election can be based any of a number of reasons. For example, intraoperatively the surgeon may not be able to access the entry location for the planned-tunnel path, and thus may need to move the entry location to ensure sufficient access. As another example, during the intraoperative procedure the surgeon may determine that the planned tunnel entry location is misaligned with the attachment location of the native ACL to the femur. Further still, during the intraoperative procedure the surgeon may determine the tunnel entry location is too close to the posterior wall of the femur, increasing the likelihood of a bone chip sometimes referred to as a “back wall blowout,” or to an existing bone tunnel as represented by a corresponding 3D cylinder model increasing the likelihood of tunnel wall collapse. Regardless of the reason for the election to change the tunnel path, in example systems the surgical controller 418 may enable the surgeon to intraoperatively select a revised-tunnel entry, a revised-tunnel exit (if needed), and thus a revised-tunnel path through the bone.

A number of systems, methods and procedures for intraoperatively selecting a revised-tunnel entry, a revised-tunnel exit, and thus a revised-tunnel path through the bone are described in Quist. It will be appreciated that a 3D cylinder model such as is described herein may be useful, when displayed for example on a display device such as display device 414 in conjunction with the corresponding 3D bone model, during such intraoperative selecting.

A number of systems, methods and procedures for computer guidance for and creation of an actual tunnel are described in Quist. Computer guidance for creation of the actual tunnel may include guidance for drilling of an initial or pilot tunnel using a drill wire (e.g., drill wire 424 (FIG. 4)), and then using the drill wire as guide wire for one or more reamers to increase the diameter of the pilot tunnel to form the full-diameter actual tunnel though the bone. In some cases the actual tunnel has a counterbore associated with the intercondylar notch to accommodate the width of the autograft, and in such cases an additional reamer may be used to create the counterbore. Computer guidance for avoiding or for boring through an existing tunnel may include displaying a 3D cylinder model such as is described herein on a display device such as display device 414 in conjunction with the corresponding 3D bone model.

### Software and Hardware

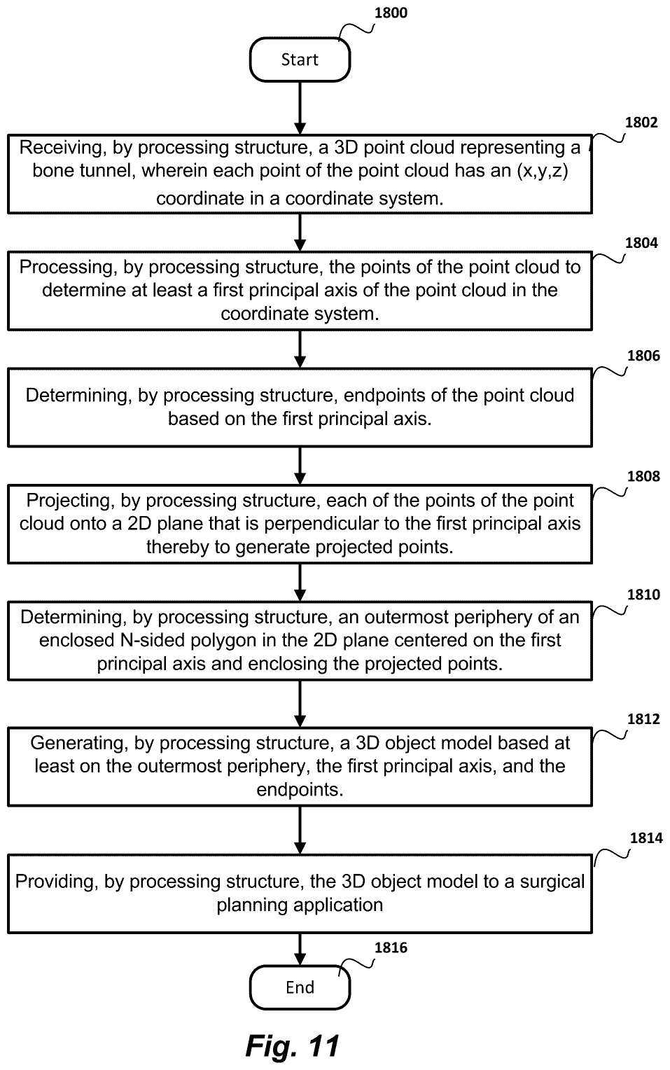

FIG. 11 shows a method, in accordance with at least some embodiments. In particular, the method starts (block 1800) and comprises: receiving, by processing structure, a 3D point cloud representing a bone tunnel, wherein each point of the point cloud has an (x,y,z) coordinate in a coordinate system (block 1802); processing, by processing structure, the points of the point cloud to determine at least a first principal axis of the point cloud in the coordinate system (block 1804); determining, by processing structure, endpoints of the point cloud based on the first principal axis (block 1806); projecting, by processing structure, each of the points of the point cloud onto a 2D plane that is perpendicular to the first principal axis thereby to generate projected points (block 1808); determining, by processing structure, an outermost periphery of an enclosed N-sided polygon in the 2D plane centered on the first principal axis and enclosing the projected points (block 1810); generating, by processing structure, a 3D object model based at least on the outermost periphery, the first principal axis, and the endpoints (block 1812); and providing, by processing structure, the 3D object model to a surgical planning application (block 1814). Thereafter, the method ends (block 1816). The example method may be implemented by computer instructions executed with the processor of computer system, such as the surgical controller 418 (FIG. 4).

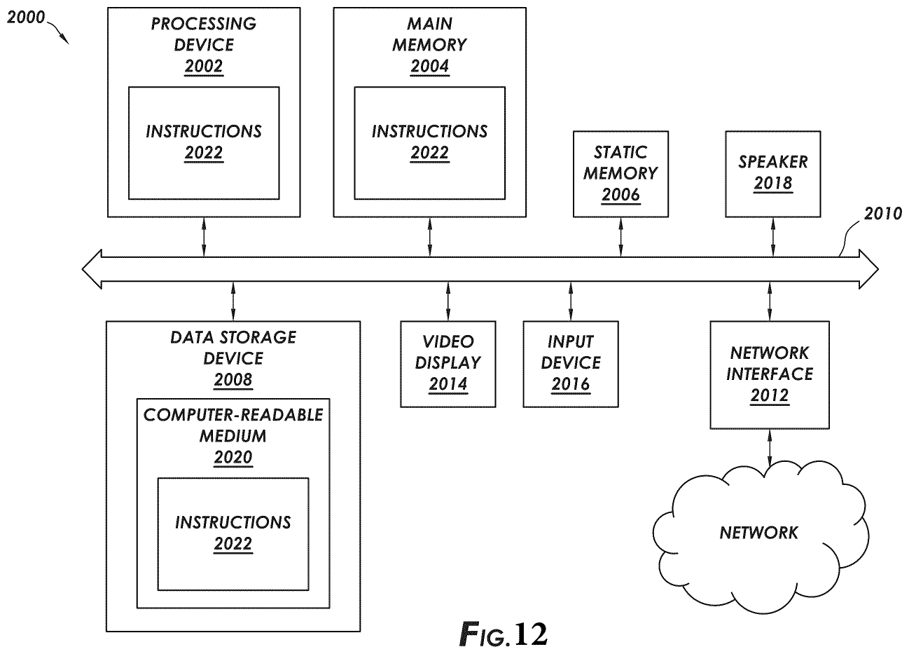

FIG. 12 shows an example computer system 2000. In one example, computer system 2000 may correspond to the surgical controller 418, a tablet device within the surgical room, or any other system that implements any or all the various methods discussed in this specification. The computer system 2000 may be connected (e.g., networked) to other computer systems in a local-area network (LAN), an intranet, and/or an extranet (e.g., device cart 402 network), or at certain times the Internet (e.g., when not in use in a surgical procedure). The computer system 2000 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 2000 includes a processing device 2002, a main memory 2004 (e.g., read-only memory (ROM), flash memory, dynamic random access memory (DRAM) such as synchronous DRAM (SDRAM)), a static memory 2006 (e.g., flash memory, static random access memory (SRAM)), and a data storage device 2008, which communicate with each other via a bus 2010.

Processing device 2002 represents one or more general-purpose processing devices such as a microprocessor, central processing unit, or the like. More particularly, the processing device 2002 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 2002 may also be one or more special-purpose processing devices such as an application specific integrated circuit (ASIC), a field programmable gate array (FPGA), a digital signal processor (DSP), network processor, or the like. The processing device 2002 is configured to execute instructions for performing any of the operations and steps discussed herein. Once programmed with specific instructions, the processing device 2002, and thus the entire computer system 2000, becomes a special-purpose device, such as the surgical controller 418.

The computer system 2000 may further include a network interface device 2012 for communicating with any suitable network (e.g., the device cart 402 network). The computer system 2000 also may include a video display 2014 (e.g., display device 414), one or more input devices 2016 (e.g., a microphone, a keyboard, and/or a mouse), and one or more speakers 2018. In one illustrative example, the video display 2014 and the input device(s) 2016 may be combined into a single component or device (e.g., an LCD touch screen).

The data storage device 2008 may include a computer-readable storage medium 2020 on which the instructions 2022 (e.g., implementing any methods and any functions performed by any device and/or component depicted described herein) embodying any one or more of the methodologies or functions described herein is stored. The instructions 2022 may also reside, completely or at least partially, within the main memory 2004 and/or within the processing device 2002 during execution thereof by the computer system 2000. As such, the main memory 2004 and the processing device 2002 also constitute computer-readable media. In certain cases, the instructions 2022 may further be transmitted or received over a network via the network interface device 2012.

While the computer-readable storage medium 2020 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

While examples have been described, variations are possible. For example, while bone tunnels characterized using 3D cylinder models are described herein, it will be appreciated that the systems and methods described herein may be used to best-fit a cartilage defect captured in cross-sectional images, such as via MRI, provided such a defect is sufficiently large to enable capture and useful visualization using MRI imaging. It will be appreciated that the extent of a cylindrical representation of a cartilage defect may be small, but could be very useful for understanding the defect so that a cartilage graft/plug could be formed and the defect itself accurately removed with little collateral damage.

While a 3D cylinder model is useful for providing surgical and planning guidance, in examples a surgeon may be provided with a point cloud representation of a tunnel that may be displayed in conjunction with the 3D bone model. The utility of being provided with just a point cloud representation of the tunnel may have a negative correlation with the amount of variability in the dimensions of the tunnel across its extent. For example, if significant bone in-growth has occurred, the point cloud will be less easily discernable by the surgeon as representative of the original cylindrical tunnel, than if little bone in-growth has occurred such that the existing tunnel is sufficiently clearly discernable as a cylinder having a first principal axis with a particular orientation, a particular diameter and a particular extent. Display for a surgeon of just a point cloud in lieu of the 3D cylinder model may be more useful when establishing a no-fly zone than when establishing a tunnel that is to be bored out for revision surgery.

As explained herein, while the existing tunnels described herein are characterized as cylinders, other best-fit shapes may have non-circular cross-sectional shapes. For example, rather than a circle for enclosing the projected points, a more appropriate best-fit shape may be a triangle, a square, a pentagon, and so forth. It may be useful to trial best-fit shapes progressively, beginning for example with sizes of triangle, and progressing through sizes of other shapes each having more and more vertices. While the standard shape of reamer, at the time of this writing, produces cylindrical tunnels, this disclosure contemplates the characterizing of existing tunnels and of tunnels to be drilled for revision reconstruction having different cross-sectional shapes than circles. Furthermore, an existing tunnel originally drilled with a circular cross-sectional shape (a 1-sided enclosed polygon) may be bored during revision surgery with a non-circular cross-sectional shape having an outermost periphery of another N-sided polygon, depending on the instrument used for reaming/boring.

Whereas embodiments described herein have been described in terms of tunnels, 3D models thereof, and 3D object models being cylindrical, this description is intended to be applicable to implementations in which such tunnels, 3D models thereof, and 3D object models being non-cylindrical.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A processor-implemented method comprising:
receiving, by processing structure, a 3D point cloud representing a bone tunnel, wherein each point of the point cloud has an (x,y,z) coordinate in a coordinate system;
processing, by processing structure, the points of the point cloud to determine at least a first principal axis of the point cloud in the coordinate system;
determining, by processing structure, endpoints of the point cloud based on the first principal axis;
projecting, by processing structure, each of the points of the point cloud onto a 2D plane that is perpendicular to the first principal axis thereby to generate projected points;
determining, by processing structure, an outermost periphery of a first enclosed N-sided polygon in the 2D plane centered on the first principal axis and enclosing the projected points;
generating, by processing structure, a 3D object model based at least on the outermost periphery, the first principal axis, and the endpoints; and
providing, by processing structure, the 3D object model to a surgical planning application.

2. The processor-implemented method of claim 1, comprising prior to the projecting:
rotating, by processing structure, each point of the point cloud to align the first principal axis of the point cloud with a z axis of the coordinate system, wherein the 2D plane that is perpendicular to the first principal axis is an x-y plane in the coordinate system.

3. The processor-implemented method of claim 1, further comprising:
determining, by processing structure, an innermost periphery corresponding to a second enclosed N-sided polygon in the 2D plane that is centered on the first principal axis and enclosed by the projected points, wherein generating the 3D object model is additionally based on the innermost periphery.

4. The processor-implemented method of claim 1, further comprising:
displaying, by the surgical planning application, the 3D object model in association with a 3D tunnel model of the bone tunnel.

5. The processor-implemented method of claim 1, further comprising, prior to receiving the point cloud:
segmenting, by processing structure, tunnel features in each of a plurality of cross-sectional images of a bone;
generating, by processing structure, a 3D tunnel model based on the segmenting; and
generating, by processing structure, the point cloud based on the 3D tunnel model.

6. The processor-implemented method of claim 5, wherein segmenting the tunnel features comprises:
generating, by processing structure, a mask representing the tunnel features for each of a plurality of the cross-sectional images.

7. The processor-implemented method of claim 1, further comprising:
receiving, by processing structure, a 3D tunnel model representing the bone tunnel; and
generating, by processing structure, the point cloud based on the 3D tunnel model.

8. The processor-implemented method of claim 1, wherein the first enclosed N-sided polygon is a first circle.

9. The processor-implemented method of claim 8, wherein the first circle is a best-fit circle.

10. The processor-implemented method of claim 8, wherein the first circle is larger in diameter by a tolerance amount than a best-fit circle.

11. The processor-implemented method of claim 10, wherein generating the 3D object model is additionally based on the best-fit circle.

12. The processor-implemented method of claim 1, wherein the surgical planning application is configurable to characterize the 3D object model as at least a portion of a no-drilling zone.

13. The processor-implemented method of claim 1, wherein the surgical planning application is configurable to characterize the 3D object model as at least a portion of a drilling zone.

14. The processor-implemented method of claim 1, wherein the 3D object model includes a plurality of peripheries including the outermost periphery.

15. A device comprising:
processing structure; and
a memory coupled to the processing structure, the memory storing instructions that, when executed by the processing structure, cause the processing structure to:
receive a 3D point cloud representing a bone tunnel, wherein each point of the point cloud has an (x,y,z) coordinate in a coordinate system;
process the points of the point cloud to determine at least a first principal axis of the point cloud in the coordinate system;
determine endpoints of the point cloud based on the first principal axis;
project each of the points of the point cloud onto a 2D plane that is perpendicular to the first principal axis thereby to generate projected points;
determine an outermost periphery of a first enclosed N-sided polygon in the 2D plane centered on the first principal axis and enclosing the projected points;
generate a 3D object model based at least on the outermost periphery, the first principal axis, and the endpoints; and
provide the 3D object model to a surgical planning application.

16. The device of claim 15, the memory storing instructions that, when executed by the processing structure, cause the processing structure to:
rotate each point of the point cloud to align the first principal axis of the point cloud with a z axis of the coordinate system, wherein the 2D plane that is perpendicular to the first principal axis is an x-y plane in the coordinate system.

17. The device of claim 15, the memory storing instructions that, when executed by the processing structure, cause the processing structure to:
determine an innermost periphery corresponding to a second enclosed N-sided polygon in the 2D plane that is centered on the first principal axis and enclosed by the projected points, wherein generating the 3D object model is additionally based on the innermost periphery.

18. The device of claim 15, wherein the processing structure is coupled to a display device, the memory storing instructions that, when executed by the processing structure, cause the processing structure to:
display, on the display device, the 3D object model in association with a 3D tunnel model of the bone tunnel.

19. The device of claim 15, the memory storing instructions that, when executed by the processing structure, cause the processing structure to:
segment tunnel features in each of a plurality of cross-sectional images of a bone;
generate a 3D tunnel model based on the segmenting; and
generate the point cloud based on the 3D tunnel model.

20. The device of claim 19, the memory storing instructions that, when executed by the processing structure, cause the processing structure to:
generate a mask representing the tunnel features for each of a plurality of the cross-sectional images.

21. The device of claim 15, the memory storing instructions that, when executed by the processing structure, cause the processing structure to:
receive a 3D tunnel model representing the bone tunnel; and
generate the point cloud based on the 3D tunnel model.

22. The device of claim 15, wherein the first enclosed N-sided polygon is a first circle.

23. The device of claim 22, wherein the first circle is a best-fit circle.

24. The device of claim 22, wherein the first circle is larger in diameter by a tolerance amount than a best-fit circle.

25. The device of claim 24, wherein generating the 3D object model is additionally based on the best-fit circle.

26. The device of claim 15, the memory storing instructions that, when executed by the processing structure, cause the processing structure to characterize the 3D object model in the surgical planning application as at least a portion of a no-drilling zone.

27. The device of claim 15, the memory storing instructions that, when executed by the processing structure, cause the processing structure to characterize the 3D object model in the surgical planning application as at least a portion of a drilling zone.

28. The device of claim 15, wherein the 3D object model includes a plurality of peripheries including the outermost periphery.
